(19) United States
(12) Patent Application Publication
(10) Pub. No.: US 2009/0270453 A1
(43) Pub. Date: Oct. 29, 2009

(54) ANTI-MANIC EFFECTIVE DOSES OF SERTINDOLE

(75) Inventor: Henriette Husum Bak-Jensen, Copenhagen S (DK)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(73) Assignee: H. Lundbeck A/S, Valby-Copenhagen (DK)

(21) Appl. No.: 12/485,691

(22) Filed: Jun. 16, 2009

Related U.S. Application Data
(63) Continuation-in-part of application No. PCT/DK08/50316, filed on Dec. 17, 2008.
(60) Provisional application No. 61/014,118, filed on Dec. 17, 2007.

(51) Int. Cl. A61K 31/454; A61P 25/18; A61P 25/28

(52) U.S. Cl. 514/323

(57) ABSTRACT

The present invention relates to uses of an anti-manic effective dose sertindole in the preparation of a pharmaceutical composition for the treatment of mania, and to methods of treating mania comprising administering said effective dose of sertindole. In separate aspects of the invention, said uses and methods are directed to treating manic episodes.

### FIELD OF THE INVENTION

The present invention relates to uses of an anti-manic effective dose of sertindole in the preparation of a pharmaceutical composition for the treatment of mania, and to methods of treating mania comprising administering said effective dose of sertindole. In separate aspects of the invention, said uses and methods are directed to treating manic episodes.

### BACKGROUND OF THE INVENTION

Throughout this application, various publications are referenced in full. The disclosures of these publications are hereby incorporated by reference into this application to describe more fully the state of the art to which this invention pertains.

An episode of mania is generally characterized by heightened mood, increased energy, and increased pressure of speech and of energy. In severe forms, delusions of grandeur or of persecution make an appearance, and in very severe cases, incoherence and a fragmented disintegration of behavior occurs (Textbook of Clinical neuropsychiatry, David. P Moore, Copyright Arnold 2001).

Cases of mania in patients where precipitating factors are not involved are covered herein. For example, bipolar disorders are neurological brain disorders, and are by far the most common cause of mania; they are characterized, in most cases, by extreme swings in mood, i.e. recurrent episodes of mania and recurrent episodes of depression throughout the lifetime of a patient. Some patients, often referred to as unipolar manic, have only manic episodes during their lifetime although such patients are more rare. Cyclothymia, which can be thought of as a mild form of bipolar disorder, is also characterized by recurrent episodes of mania and recurrent episodes of depression, but these are milder in intensity. Lastly, schizoaffective disorder, displays a distinctive overall course, and patients suffering therefrom are chronically psychotic and in addition experience episodes of mania and episodes of depression (See Diagnostic and Statistical Manual of Mental Disorders, Fourth Edition, American Psychiatric Association, 1994 (DSM-IV)). Bipolar disorders are life-threatening conditions, since patients diagnosed with bipolar disorder have an estimated suicide risk that is 15 times higher than in the general population (Harris and Barraclough, British Journal of Psychiatry, 1997, 170, 205-228).

It should also be mentioned that Alzheimer's disease, neurosyphilis, stroke, midbrain infarctions, thalamic infarctions, bilateral thalamic infarction, Huntington's disease, Sydenham's chorea, Chorea gravidarum, Cushing's disease, thyrotoxicosis, cerebral tumors, multiple sclerosis, systemic lupus erythematosus, vitamin B12 deficiency, hepatic encephalopathy, uremia, Creutzfeldt-Jakob disease, ictal mania and post-ictal psychoses are all clinical indications or factors that have been associated with occurrence of mania in patients (Textbook of clinical neuropsychiatry, David. P Moore, Copyright Arnold 2001).

Psychiatric research on bipolar disorders has focused on the role of neurobiology, but a clear organic cause has not been found. At present, bipolar disorders are treated by maintaining patients on mood-stabilizing therapy (mainly lithium or anti-epileptics) combined with adjunctive treatment with antidepressants (tricyclic antidepressants or SSRIs) when the patients relapse into a depressive episode, or combined with anti-psychotics when the patients relapse into a manic episode (Liebermann and Goodwin, Curr. Psychiatry Rep. 2004, 6, 459-465). It is believed to be important to effectively treat the manic episodes as the occurrence of these may contribute to a poorer prognosis with regard to relapse as well as with regard to severity of symptoms in later manic or depressive episodes (Kukopulos, et al. Compr. Psychiatry 1983, 24, 249-258).

However, the use of lithium has a number of disadvantages, including the importance of establishing and maintaining an appropriate concentration of lithium in the blood, as well as being associated with a plethora or physiological conditions including hypothyroidism, tremors, dry mouth, weight gain, increased frequency of urination, nausea, impotence, decreased libido, diarrhea, kidney abnormalities, loss of appetite, visual impairment, seizures and arrhythmias. Additionally, the use of the other mainstay drug, valproic acid (otherwise known as valproate), is associated with hepatic dysfunction. Thus, a need exists to develop novel and improved therapeutic treatments for manic episodes, such as for instance manic episodes in bipolar disorders.

To discover new treatments, the pharmaceutical industry frequently employs certain strategies such as screening compounds against relevant animal models predictive of a particular medical indication. However, since there is no animal model that covers the full symptomatic spectrum of bipolar disorders, separate animal models of either manic or depression symptoms are employed instead (Einat, Beha. Genet. 2007, 37, 244-255; and Machado-Vieira, et al., J.C. Prog. Neuropsychopharmacol. Biol. Psychiatry 2004, 28, 209-224). Behavioral models of mania are often based on assessing increased locomotor activity as a means of modeling the core manic symptoms of excessive activity, i.e. decreased need for sleep, increased sexual drive, pressure of speech and racing thoughts (Einat, Behav Genet. 2007, 37, 244-255).

Behavioral sensitization is a process whereby repeated intermittent administration of a psychostimulant results in a time dependent, enduring, and progressively greater or more rapid behavioral response (Robinson and Berridge, Brain Res. Rev. 1993, 18, 247-291). The major neural circuit believed to be involved in behavioral sensitization is the mesolimbic dopamine pathway (Robinson and Becker, Brain Res. 1986, 396, 157-198). In bipolar patients, affective episodes show a progressive recurrence and an increased severity with time. This phenomenon has been interpreted as a process of sensitization (Kessing, et al. J. Affective Disorders 1998, 47, 31-39). Furthermore, patients with first-episode manic or schizophrenic psychosis were reported not to demonstrate a sensitized response following a second dose of amphetamine (AMPH), in contrast to the sensitized response that is seen in normal volunteers (Strakowski, et al., Biol. Psychiatry 1997, 42, 749-755). These results imply that manic/psychotic patients may already be sensitized, and support on its face the validity of the sensitization model in the context of mania.

Lithium and lamotrigine are cornerstone treatments in the acute treatment of mania. Consequently, positive effects of treatment with lithium and lamotrigine are central to the predictive validity of an animal model of mania (Einat, et al. Psychopharmacol. Bull. 2003, 37, 47-63). In agreement with previous reports (Lamberty, et al. Epilepsy Behav. 2001, 2, 454-459), lithium and lamotrigine significantly reduced the amphetamine (AMPH)+chlordiazepoxide (CDP) induced locomotor hyperactivity in rats. These effects were not due to unspecific motor effects, as basal locomotor activity was not reduced by these treatments. Mood stabilizers such as valproate and carbamazepine, that are also used in the acute and prophylactic treatment of bipolar disorders, are also known to be effective in this model (Lamberty, et al. Epilepsy Behav. 2001, 2, 454-459). To this end, the sensitized AMPH+CDP response model appears to have predictive validity and this model appears to be appropriate to detect compounds with anti-mania potential.

Additionally, it has been shown that lithium, lamotrigine, valproate and carbamazepine dose-dependently abolish the sensitized response to an acute AMPH challenge in mice (Husum, H. 8^{th} World Congress of Biological Psychiatry 2005, poster no. 32.06). Consequently, the sensitized AMPH response model shows good predictive validity and is an appropriate model to use to detect compounds with antimanic-like properties.

Sertindole, chemically named 5-chloro-1-(4-fluorophenyl)-3-(1-(2-(2-imidazolinon-1-yl) ethyl-4-piperidyl-1H-indole, is an antipsychotic drug with high affinity for serotonin 5-HT₂, dopamine D₂ and α₁-adrenergic receptors. Sanchez et al., Drug Dev. Res. 1991, 22, 239-250; and Arnt and Skarsfeldt Neuropsychopharmacol. 1998, 18, 63-101. Sertindole is disclosed in Re. 34,299, and its antipsychotic activity is disclosed in U.S. Pat. No. 5,112,838. A method of manufacturing sertindole is disclosed in U.S. Pat. No. 6,335,463.

Most research directed at the therapeutic effectiveness of sertindole has focused on its use in the treatment of schizophrenia. See, e.g. U.S. Pat. No. 5,112,838; Brown et al., Pharmacotherapy 1993, 18, 69-83; Samara and Granneman, R. Clin. Pharmacol. & Therapeutics 1996, 59, 187; and Tamminga et al., International Clin. Psychopharmacol. 1997, 12 (suppl. 1), S29-S35. Sertindole may also be effective in the treatment of other disorders, such as: psychosis, including drug induced psychosis (U.S. Pat. No. 5,238,945); anxiety (U.S. Pat. No. 5,439,922); memory impairment (U.S. Pat. No. 5,444,073); substance dependency (U.S. Pat. No. 5,462,948); and depression, hypertension, and extrapyramidal side effects of other antipsychotic drugs (U.S. Pat. No. 5,703,087).

To evaluate its potential to treat bipolar disorders, sertindole was tested in the sensitized AMPH+CDP and the sensitized AMPH response models. Consistent with the results obtained from the experiments involving known compounds, the results of the animal model studies with sertindole, described herein, support the hypothesis that sertindole has the potential to treat mania in a human subject, e.g. mania in bipolar disorders.

In the U.S., certain marketed anti-psychotic drugs which are used to treat schizophrenia have also been approved by US Food and Drug Administration (FDA) for the treatment of manic or mixed episodes of bipolar disorders. As an example, Abilify® is currently approved in the US to treat schizophrenia as well as the manic episodes of bipolar disorders. For adults with schizophrenia who are first starting Abilify®, dosing typically starts at 10 mg to 15 mg once daily. For the treatment of bipolar disorder, most adults start with a daily dose of 15 mg. http://www.accessdata.fda.gov/scripts/cder/ob/default.cfm. Likewise, the recommended starting dose of Geodon® for schizophrenia is 20 mg twice per day while the recommended starting dose of Geodon® for bipolar disorder is 40 mg twice per day. Other anti-psychotics approved for mania typically use the normal or typical antipsychotic dose to achieve that effect.

While human clinical studies demonstrate the effectiveness of sertindole to treat schizophrenic patients at doses of about 12-24 mg/day (Daniel D, et al., 34th Annual Meeting of the American College of Neuropsychopharmacology, San Juan, Puerto Rico, December 1995), applicants believe that the instant experiments indicate that a lower effective dose of Sertindole to treat mania and/or bipolar disorders may be needed as compared to the effective dose needed to elicit an anti-psychotic effect. A recent paper (Olsen, et al. Eur. J. Pharmacol. 2008, 584, 328-327) describes the how pharmacokinetic-pharmacodynamic (PK-PD) modeling applies to the anti-psychotics and the conditional avoidance response paradigm by evaluating the degree of correlation between PK/PD predictions of therapeutically effective steady-state levels for various anti-psychotics. The authors determined that predictions of therapeutically effective steady-state levels for sertindole were about 3-4 times too high. Accordingly, applicants believe this anti-manic effective dose would enable the maintenance dose needed to treat mania to be about 3-4 times lower that than the maintenance dose needed to treat the symptoms of psychosis as seen in, for example, schizophrenic patients.

### SUMMARY OF THE INVENTION

The present invention is directed to a use of sertindole in the preparation of a pharmaceutical composition for the treatment of mania in a subject, wherein the composition comprises an anti-manic effective dose of sertindole.

Additionally, the subject invention is directed to a use of sertindole in the preparation of a pharmaceutical composition for the treatment of bipolar disorders in a subject, wherein the composition comprises an anti-manic effective dose of sertindole.

Further, the present invention relates to a use of sertindole in the preparation of a pharmaceutical composition for the treatment mania in a subject suffering from any one of Alzheimer's disease, neurosyphilis, stroke, midbrain infarctions, thalamic infarctions, bilateral thalamic infarction, Huntington's disease, Sydenham's chorea, Chorea gravidarum, Cushing's disease, thyrotoxicosis, cerebral tumors, multiple sclerosis, systemic lupus erythematosus, vitamin B12 deficiency, hepatic encephalopathy, uremia, Creutzfeldt-Jakob disease, ictal mania and post-ictal psychoses, wherein the composition comprises an anti-manic effective dose of sertindole.

Furthermore, the invention relates to sertindole for the treatment of bipolar disorders in a subject. Additionally, the inventions relates to sertindole for the treatment of mania in a subject.

The present invention also relates to use of sertindole in the preparation of a pharmaceutical composition for the treatment of mania in a subject suffering from a bipolar disorder, wherein the composition comprises an anti-manic effective dose of sertindole.

The present invention also relates to use of sertindole in the preparation of a pharmaceutical composition for the treatment of mania in a subject suffering from Alzheimer's disease, wherein the composition comprises an anti-manic effective dose of sertindole.

The present invention also relates to use of sertindole in the preparation of a pharmaceutical composition for the treatment of mania in a subject suffering from neurosyphilis, wherein the composition comprises an anti-manic effective dose of sertindole.

The present invention also relates to use of sertindole in the preparation of a pharmaceutical composition for the treatment of mania in a subject suffering from stroke, wherein the composition comprises an anti-manic effective dose of sertindole.

The present invention also relates to use of sertindole in the preparation of a pharmaceutical composition for the treatment of mania in a subject suffering from midbrain infarctions, wherein the composition comprises an anti-manic effective dose of sertindole.

The present invention also relates to use of sertindole in the preparation of a pharmaceutical composition for the treatment of mania in a subject suffering from thalamic infarctions, wherein the composition comprises an anti-manic effective dose of sertindole.

The present invention also relates to use of sertindole in the preparation of a pharmaceutical composition for the treatment of mania in a subject suffering from bilateral thalamic infarction, wherein the composition comprises an anti-manic effective dose of sertindole.

The present invention also relates to use of sertindole in the preparation of a pharmaceutical composition for the treatment of mania in a subject suffering from Huntington's disease, wherein the composition comprises an anti-manic effective dose of sertindole.

The present invention also relates to use of sertindole in the preparation of a pharmaceutical composition for the treatment of mania in a subject suffering from Sydenham's chorea, wherein the composition comprises an anti-manic effective dose of sertindole.

The present invention also relates to use Of sertindole in the preparation of a pharmaceutical composition for the treatment of mania in a subject suffering from Chorea gravidarum, wherein the composition comprises an anti-manic effective dose of sertindole.

The present invention also relates to use of sertindole in the preparation of a pharmaceutical composition for the treatment of mania in a subject suffering from Cushing's disease, wherein the composition comprises an anti-manic effective dose of sertindole.

The present invention also relates to use of sertindole in the preparation of a pharmaceutical composition for the treatment of mania in a subject suffering from thyrotoxicosis, wherein the composition comprises an anti-manic effective dose of sertindole.

The present invention also relates to use of sertindole in the preparation of a pharmaceutical composition for the treatment of mania in a subject suffering from cerebral tumors, wherein the composition comprises an anti-manic effective dose of sertindole.

The present invention also relates to use of sertindole in the preparation of a pharmaceutical composition for the treatment of mania in a subject suffering from multiple sclerosis, wherein the composition comprises an anti-manic effective dose of sertindole.

The present invention also relates to use of sertindole in the preparation of a pharmaceutical composition for the treatment of mania in a subject suffering from systemic lupus erythematosus, wherein the composition comprises an anti-manic effective dose of sertindole.

The present invention also relates to use of sertindole in the preparation of a pharmaceutical composition for the treatment of mania in a subject suffering from vitamin B12 deficiency, wherein the composition comprises an anti-manic effective dose of sertindole.

The present invention also relates to use of sertindole in the preparation of a pharmaceutical composition for the treatment of mania in a subject suffering from hepatic encephalopathy, wherein the composition comprises an anti-manic effective dose of sertindole.

The present invention also relates to use of sertindole in the preparation of a pharmaceutical composition for the treatment of mania in a subject suffering from uremia, wherein the composition comprises an anti-manic effective dose of sertindole.

The present invention also relates to use of sertindole in the preparation of a pharmaceutical composition for the treatment of mania in a subject suffering from Creutzfeldt-Jakob disease, wherein the composition comprises an anti-manic effective dose of sertindole.

The present invention also relates to use of sertindole in the preparation of a pharmaceutical composition for the treatment of mania in a subject suffering from ictal mania and post-ictal psychoses, wherein the composition comprises an anti-manic effective dose of sertindole.

In one embodiment, a subject is a human, such as male or female human, child, adult or elderly.

The present invention is also directed to a kit comprising (a) an anti-manic effective dose of sertindole in one or more unit dosages; (b) a finished pharmaceutical container containing said unit doses; and (c) a label stating that said dosages can be administered to treat bipolar disorders.

Furthermore, the subject invention is directed to a method of marketing an anti-manic effective dose of sertindole by marketing, advertising, or selling sertindole for the treatment of bipolar disorders.

Additionally, the subject invention is directed to a method of treating bipolar disorders comprising administering to a subject in need thereof an anti-manic effective dose of sertindole.

The subject invention is directed to a method of treating mania comprising administering to a subject in need thereof an anti-manic effective dose of sertindole.

Further, the present invention relates to a method of treating mania in a subject suffering from a bipolar disorder comprising administering to the subject in need thereof an anti-manic effective dose of sertindole.

In separate aspects of the invention, the bipolar disorder is bipolar I disorder, bipolar II disorder, cyclothymia, or bipolar NOS.

Further, the present invention relates to a method of treating mania in a subject suffering from any one of Alzheimer's disease, neurosyphilis, stroke, midbrain infarctions, thalamic infarctions, bilateral thalamic infarction, Huntington's disease, Sydenham's chorea, Chorea gravidarum, Cushing's disease, thyrotoxicosis, cerebral tumors, multiple sclerosis, systemic lupus erythematosus, vitamin B12 deficiency, hepatic encephalopathy, uremia, Creutzfeldt-Jakob disease, ictal mania and post-ictal psychoses comprising administering to the subject in need thereof an anti-manic effective dose of sertindole.

The present invention also relates to a method of treating mania in a subject suffering from a bipolar disorder comprising administering an anti-manic effective dose of sertindole.

The present invention also relates to a method of treating mania in a subject suffering from Alzheimer's disease comprising administering an anti-manic effective dose of sertindole.

The present invention also relates to a method of treating mania in a subject suffering from neurosyphilis, wherein the composition comprises an anti-manic effective dose of sertindole.

The present invention also relates to a method of treating mania in a subject suffering from stroke comprising administering an anti-manic effective dose of sertindole.

The present invention also relates to a method of treating mania in a subject suffering from midbrain infarctions comprising administering an anti-manic effective dose of sertindole.

The present invention also relates a method of treating mania in a subject suffering from thalamic infarctions comprising administering an anti-manic effective dose of sertindole.

The present invention also relates to a method of treating mania in a subject suffering from bilateral thalamic infarction comprising administering an anti-manic effective dose of sertindole.

The present invention also relates to a method of treating mania in a subject suffering from Huntington's disease comprising administering an anti-manic effective dose of sertindole.

The present invention also relates to a method of treating mania in a subject suffering from Sydenham's chorea comprising administering an anti-manic effective dose of sertindole.

The present invention also relates to a method of treating mania in a subject suffering from Chorea gravidarum comprising administering an anti-manic effective dose of sertindole.

The present invention also relates to a method of treating mania in a subject suffering from Cushing's disease comprising administering an anti-manic effective dose of sertindole.

The present invention also relates to a method of treating mania in a subject suffering from thyrotoxicosis comprising administering an anti-manic effective dose of sertindole.

The present invention also relates to a method of treating mania in a subject suffering from cerebral tumors comprising administering an anti-manic effective dose of sertindole.

The present invention also relates to a method of treating mania in a subject suffering from multiple sclerosis comprising administering an anti-manic effective dose of sertindole.

The present invention also relates to a method of treating mania in a subject suffering from systemic lupus erythematosus comprising administering an anti-manic effective dose of sertindole.

The present invention also relates to a method of treating mania in a subject suffering from lupus erythematosus comprising administering an anti-manic effective dose of sertindole.

The present invention also relates to a method of treating mania in a subject suffering from vitamin B12 deficiency comprising administering an anti-manic effective dose of sertindole.

The present invention also relates to a method of treating mania in a subject suffering from hepatic encephalopathy comprising administering an anti-manic effective dose of sertindole.

The present invention also relates to a method of treating mania in a subject suffering from uremia comprising administering an anti-manic effective dose of sertindole.

The present invention also relates to a method of treating mania in a subject suffering from Creutzfeldt-Jakob disease comprising administering an anti-manic effective dose of sertindole.

The present invention also relates to a method of treating mania in a subject suffering from ictal mania and post-ictal psychoses comprising administering an anti-manic effective dose of sertindole.

In a further aspect the present invention relates to combination of sertindole with a further medicament, such as lithium, valproate, lamotrigine or carbamazepine.

### BRIEF DESCRIPTION OF THE DRAWINGS

FIG. 1 presents the data from the sensitized AMPH+CDP response model using lithium.

FIG. 2 displays the data from the sensitized AMPH+CDP response model using sertindole.

FIG. 3 presents the data from the sensitized AMPH+CDP response model using lamotrigine.

FIG. 4 displays the data from the sensitized AMPH+CDP response model using citalopram.

FIG. 5 presents the data from the sensitized AMPH response model using lithium.

FIG. 6 presents the data from the sensitized AMPH response model using sertindole.

FIG. 7 presents the data from the sensitized AMPH response model using citalopram.

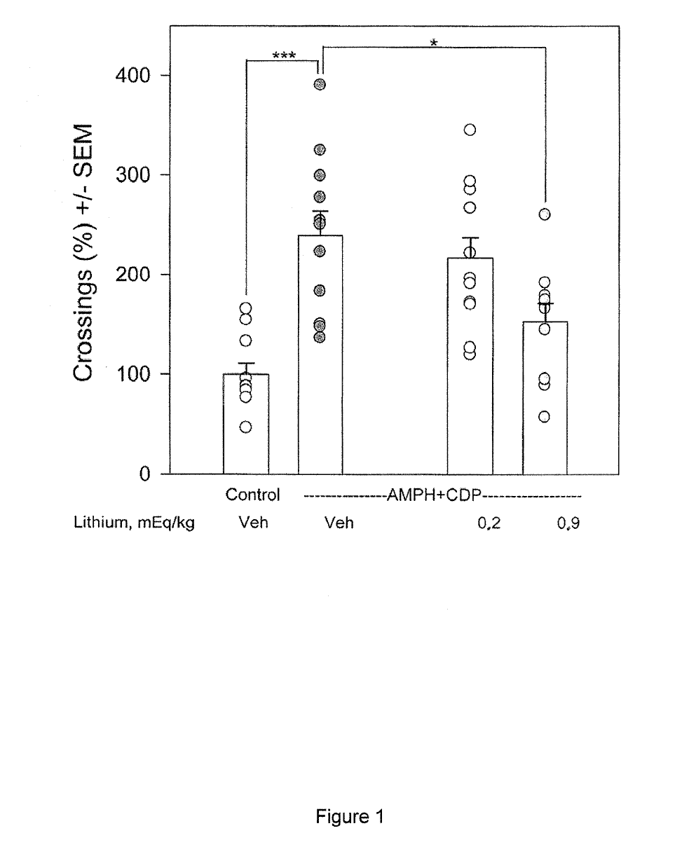

FIGS. 1-7 display the crossings of light-beams as a measure of locomotor activity over a definite test-period as percentage of the control group-level that is set to 100%. The bars show the mean±SEM for each group and the dots show the activity of individual rats.

### DETAILED DESCRIPTION OF THE INVENTION

The present invention is based on the discovery that sertindole is active in the sensitized AMPH+CDP and sensitized AMPH animal models, and thus, has the potential to treat mania and bipolar disorders, such as mania in bipolar disorders. The instant experiments indicate that a lower effective dose of sertindole to treat mania and/or bipolar disorders may be needed as compared to the effective dose needed to elicit an anti-psychotic effect.

The invention is explained in greater detail below, but this description is not intended to be a detailed catalog of all the different ways in which the invention may be implemented, or all the features that may be added to the instant invention.

### Definitions

As used herein, the term “sertindole” comprises the free base or pharmaceutically acceptable salts of 5-chloro-1-(4-fluorophenyl)-3-(1-(2-(2-imidazolinon-1-yl) ethyl-4-piperidyl-1H-indole, in either crystalline or amorphous form, as well as solvates such as hydrates. Doses of sertindole as specified herein refer to the free base form unless otherwise specified. “A once daily dosage form containing 1-24 mg of sertindole” thus means “a once daily dosage form containing 1-24 mg of sertindole calculated as the free base”.

The pharmaceutically acceptable acid addition salts of the compound may be formed with non-toxic organic or inorganic acids in an aqueous miscible solvent, such as acetone or ethanol, with isolation of the salt by concentration and cooling or an excess of the acid in aqueous immiscible solvent, such as ethyl ether or chloroform, with the desired salt separating directly.

Exemplary of such organic salts are those with maleic, fumaric, benzoic, ascorbic, embonic, succinic, oxalic, bis methylene-salicylic, methanesulfonic, ethanedisulfonic, acetic, propionic, tartaric, salicylic, citric, glucomic, lactic, malic, mandelic, cinnamic, citraconic, aspartic, stearic, palmitic, itaconic, glycolic, p-amino-benzoic, glutamic, benzene sulfonic and theophylline acetic acids as well as the 8-halotheophyllines, for example 8-bromo-theophylline. Exemplary of such inorganic salts are those with hydrochloric, hydrobromic, sulfuric, sulfamic, phosphoric and nitric acids. Of course, these salts may also be prepared by the classical method of double decomposition of appropriate salts, which is well-known to the art.

When it is desired to isolate sertindole in the form of the free base, this may be done according to conventional procedures, such as by dissolving the isolated or un-isolated salt in water, treating with a suitable alkaline material, extracting the liberated free base with a suitable organic solvent, optionally drying the extract with a suitable drying agent prior to evaporating the extract to dryness to effect isolation of the free basic amine. The extract may optionally be subjected to fractional distillation.

The term “bipolar disorders”, as referred to herein, is well known to those of skill in the art and is defined in art-recognized medical texts such as the Diagnostic and Statistical Manual of Mental Disorders, Fourth Edition, American Psychiatric Association, 1994 (DSM-IV), which is incorporated herein by reference in its entirety. Accordingly, the term “bipolar disorders” is used to include the following four types of illnesses: bipolar I disorder, bipolar II disorder, cyclothymia, and bipolar NOS.

As used herein, the term “treating” refers to reversing, alleviating, inhibiting the progress of, preventing the reoccurrence of, or preventing the disorder or condition to which such term applies, or preventing or alleviating one or more symptoms of such disorder or condition. The term “treatment”, as used herein, refers to the act of treating, as “treating” is defined immediately above. The terms “treat”, “treatment”, and “treating” include preventive (e.g., prophylactic) and palliative treatment or the act of providing preventive or palliative treatment.

As used herein, the term “anti-manic effective dose” refers to a dose of sertindole sufficient to treat bipolar disorders, mania symptoms of bipolar disorders (including acute mania, hypomania and depression, or a combination thereof); sufficient to treat mood stabilization; sufficient to prevent relapse into bipolar episodes; or sufficient to a treat suicidal thoughts and tendencies. If is believed that this dose would be lower that the “effective dose” of sertindole to elicit an anti-psychotic effect in a patient.

As used herein, the term “maintenance dose” refers to the systematic dosage at a level that maintains the anti-manic effects of sertindole in a patient after the initial titration period.

The present invention is further directed to a use of sertindole in the preparation of a pharmaceutical composition for the treatment of symptoms of bipolar disorder selected from the group consisting of acute mania and depression, wherein the composition comprises an anti-manic effective dose of sertindole. The present invention is also directed to sertindole for the treatment of bipolar disorders in a subject and to sertindole for the treatment of mania in a subject. Furthermore, the invention relates to a method of treating the symptoms of bipolar disorder selected from the group consisting of acute mania and depression in a subject comprising administering an anti-manic effective dose of sertindole.

The “symptoms of bipolar disorder selected from the group consisting of acute mania and depression” refer to, respectively, one or more symptoms that may be associated with a manic episode or a depression episode, as the case may be, of bipolar disorder.

As used herein, an “anti-psychotic” refers to the typical or first generation drugs to treat psychosis such as haloperidol and chlorpromazine, as well as the atypical anti-psychotics such as aripiprazole, clozapine, ziprasidone, risperidone, queitiapine and olanzapine.

Additionally, the invention further provides, but is not limited to certain aspects and embodiments of the present invention as described below:

In one aspect the present invention relates use of sertindole in the preparation of a pharmaceutical composition for the treatment of mania in a subject, wherein the composition comprises an anti-manic effective dose of sertindole.

In a still further aspect the invention relates to use of sertindole in the preparation of a pharmaceutical composition for the treatment of mania in a subject suffering from a bipolar disorder, wherein the composition comprises an anti-manic effective dose of sertindole.

It is to be understood that bipolar disorders are neurological brain disorders and comprises bipolar I disorder, bipolar II disorder, cyclothymia, and bipolar disorder NOS. Throughout the specification and whenever bipolar disorders are specified it is to be understood that any one of bipolar I disorder, bipolar II disorder, cyclothymia, and bipolar disorder NOS may be the subject of a particular embodiment. Thus, as an example, an embodiment of the present invention is directed to the use of sertindole in the preparation of a pharmaceutical composition for the treatment of mania in a subject suffering from bipolar I disorder, wherein the composition comprises an anti-manic effective dose of sertindole.

In a further embodiment, the bipolar disorder is bipolar II disorder. In yet another embodiment, the bipolar disorder is cyclothymia. In a further embodiment, the bipolar disorder is bipolar disorder NOS.

Any route of administration as described herein after, such as the oral route, may be applied to administer a pharmaceutical composition comprising sertindole, wherein the composition comprises an anti-manic effective dose of sertindole.

In a further embodiment, a pharmaceutical composition in the context of the invention is to be administered as a once daily oral unit dosage form, wherein the composition comprises an anti-manic effective dose of sertindole.

When the pharmaceutical composition comprising sertindole is administered by the oral route as a once daily oral unit dosage form it contains between about 1 mg and about 24 mg of sertindole, such as the free crystalline base of sertindole, wherein the composition comprises an anti-manic effective dose of sertindole.

In one embodiment, the first initial dose of a pharmaceutical composition in the context of the present invention is about 1 mg sertindole/day. In another embodiment, the first maintenance dose is reached by increasing the initial dose by about 1 mg/day. In a further embodiment of the present invention, the next maintenance dose is reached by increasing the current dose by about 1 mg/day every 4-5 days until a maintenance dose, or the maximum dose (about 24 mg/kg) is reached.

In another embodiment, the established sertindole maintenance dose is changed by about 1 mg/day to establish a new maintenance dose. For example, a sertindole maintenance dose can be increased from about 1 mg/day to about 3 mg/day and, after one or more days, increased again by about 1 mg/day to about 24 mg/day. Thus, the dose of sertindole can be increased or decreased until an optimal dose (the new maintenance dose) or the maximum dose is reached.

In one embodiment, the maintenance dose is from about 1-12 mg/day. In another embodiment, the maintenance dose is from about 2-11 mg/day. In another embodiment, the maintenance dose is from about 3-10 mg/day. In another embodiment, the maintenance dose is from about 4-9 mg/day. In another embodiment, the maintenance dose is from about 5-8 mg/day. In another embodiment, the maintenance dose is from about 6-7 mg/day.

In one embodiment, the anti-manic effective dose of sertindole is a maintenance dose. In one embodiment, the subject is a human patient diagnosed with mania or a bipolar disorder.

It is to be understood that throughout the specification sertindole may be used in any of its forms, such as without limitation the free base or pharmaceutically acceptable salts thereof, in either crystalline or amorphous form, as well as solvates such as hydrates. Throughout the specification one embodiment of sertindole is the free base form, such as the crystalline base. It is intended that the free base can be used in any aspect or embodiments of the present invention.

A pharmaceutical composition comprising sertindole may also be administered together with other medicines, such as lithium, valproate, lamotrigine and carbamazepine, as ad on treatment or combined into the same composition, such as a mixture.

In one embodiment, sertindole is administered in combination with lithium. In a further embodiment, sertindole is administered in combination with valproate. In a still further embodiment, sertindole is administered in combination with lamotrigine. In a further embodiment, sertindole is administered in combination with carbamazepine. In a still further embodiment, sertindole is administered in combination with lithium and one or more of valproate, lamotrigine and carbamazepine.

In another embodiment, the subject to be treated does not respond sufficiently to current therapies with atypical anti-psychotics. In one embodiment, the anti-psychotic is olanzapine. In one embodiment, the anti-psychotic is risperidone. In one embodiment, the anti-psychotic is clozapine. In one embodiment, the anti-psychotic is haloperidol.

In a further aspect of the present invention a kit is provided comprising (a) an anti-manic effective dose of sertindole in one or more unit dosages; (b) a finished pharmaceutical container containing said unit doses; and (c) a label stating that said dosages can be administered to treat mania.

In a further aspect of the present invention a kit is provided comprising (a) an anti-manic effective dose of sertindole in one or more unit dosages; (b) a finished pharmaceutical container containing said unit doses; and (c) a label stating that said dosages can be administered to treat bipolar disorders.

In a further aspect the present invention relates to a method of treating mania comprising administering to a subject in need thereof an anti-manic effective dose of sertindole.

In a still further aspect the present invention relates to a method of treating bipolar disorders comprising administering to a subject in need thereof an anti-manic effective dose of sertindole.

In a further aspect the present invention relates to a method of treating mania in a subject suffering from a bipolar disorder comprising administering to a subject in need thereof an anti-manic effective dose of sertindole.

Another aspect of the present invention relates to a method of marketing sertindole by marketing, advertising, or selling sertindole for the treatment of bipolar disorders. The marketing may be directed to, for example, doctors (such as psychiatrists) treating human subjects suffering from bipolar disorders. The marketing step may comprise the step of including a statement in the labeling of a pharmaceutical product or composition containing sertindole that the product or composition can be used to treat bipolar disorders in a human subject.

Sertindole may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. Examples of liquid carriers are syrup, peanut oil, olive oil, phospholipids, fatty acids, fatty acid amines, polyoxyethylene and water. Examples of solid carriers are lactose, terra alba, sucrose, cyclodextrin, talc, agar, pectin, acacia, stearic acid and lower alkyl ethers of cellulose corn starch, potato starch, talcum, magnesium stearate, gelatine, lactose, gums, and the like. Any other adjuvants or additives usually used for such purposes such as colorings, flavorings, preservatives etc. may be used provided that they are compatible with the active ingredients.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

As used herein a unit dose is the amount of a medication administered to a patient in a single dose. Unit-dose packaging is the packaging of a single dose in a non-reusable container. It is increasingly used in hospitals, nursing homes, etc. Medications in unit-dose packaging are easily identifiable and can be returned to the pharmacy if the medication is discontinued.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants. For parenteral routes such as intravenous, intrathecal, intramuscular and similar administration, typical doses are in the order of half the dose employed for oral administration.

The present invention also provides a process for making a pharmaceutical composition comprising admixing an anti-manic effective dose of a crystalline base form or solvate of sertindole and optionally a pharmaceutically acceptable carrier.

### EXPERIMENTAL SECTION

The following procedures are representative of the methods and materials which can be used to perform the sensitized AMPH+CDP and sensitized AMPH response models. However, one skilled in the art will recognize that the procedures and parameters described below are not meant to be rigid and that one skilled in the art would recognize the appropriate modification to a certain procedure.

### Example 1

### Sensitized AMPH+CDP Response Model

Subjects: Male Wistar rats weighing about 150-200 g were housed 2-4 rats per cage in makrolon cages (about 20×about 35 cm) equipped with sawdust and with one plastic house for enrichment. The animals were kept at room temperature (about 20° C.±2) in about 12-hour light/dark cycle (lights on at about 06:00) with free access to food and tap water. The rats were allowed to acclimatize to the animal facility premises for about 5-9 days prior to the initiation of experiments. The animals were taken to the experimental room on the day before the experiment and weighed.

Drugs: All drugs are listed as mg free base/kg with the exception of lithium (mEq/kg). Drug solutions were titrated into a pH range of about 4.5-7.0 for subcutaneous (s.c.) administration. To induce hyperactivity, amphetamine sulphate (about 0.9 mg/kg in about 0.9% NaCl, about 1 ml/kg) followed by CDP (7-Chloro-2-(methylmino)-5-phenyl-3h-1,4-benzodiazepine-4-oxide, about 8.9 mg/kg in about 10% hydroxypropyl-beta-cyclodextrin) was administrated about 35 minutes before the test. A parallel set of control animal received two vehicle injections. Lithium chloride (about 0.2-0.9 mEq/kg) was dissolved in distilled water isosmotically supplemented with NaCl, and administrated about 210 minutes before testing. Lamotrigine (6-(2,3-Dichloro-phenyl)-[1,2,4]-triazine-3,5-diamine, about 2.5-80 mg/kg) was dissolved in about 10% hydroxypropyl-beta-cyclodextrin and administrated about 30 minutes before the test.

Test procedure: For the assessment of locomotor activity, makrolon cages (about 20×about 35 cm) with a thin layer of standard bedding material were placed in a U-frame equipped longitudinally with 4 infrared light sources and photocells placed about 4 cm above the bottom of the cage. All experiments were conducted under normal light conditions. The animals were placed individually in test boxes and the assessment of locomotor activity was immediately begun. During the test session, locomotor activity was recorded as crossings of infrared light beams and total locomotor activity was the accumulated number of crossings over the approximately 120-min period. The recording of a motility count required the crossing of two adjacent light beams, thus avoiding counts induced by stationary movements of the rat.

Data Analysis: The average total basal locomotor activity of the control groups in the different experiments varied between 322-516 light beam crossings (see Table 1). Due to this variation, the results in each experiment were normalized to percentage activity relative to the average total locomotor activity of control group that was set as 100%. Statistical analysis of differences in total locomotor activity (normalized data) among the various treatment groups was carried out using a two-way ANOVA with factors treatment (AMPH+CDP vs. vehicle) and drug (drug vs. vehicle). In case of a significant interaction, a post hoc Student-Newman-Keuls Method was used for multiple comparisons. A probability level of 0.05 was considered significant.

Crossings of light-beams as a measure of locomotor activity over about a 120 min test-period is shown as percentage of the control group-level that is set to 100%. The bars show the mean±SEM for each group and the dots show the activity of individual rats (n=10-12). Administration of amphetamine (about 0.9 mg/kg, s.c.) and chlordiazepoxide (about 8.9 mg/kg, s.c.) produced a significant increase in locomotor activity (***p<0.001 vs. control group (O) in all experiments).

FIG. 1: Pre-treatment with lithium (about 210 min, s.c.) reduced the induced hyperactivity. The effect was significant at about 0.9 mg/kg (*p<0.05 vs. AMPH−CDP group ()).

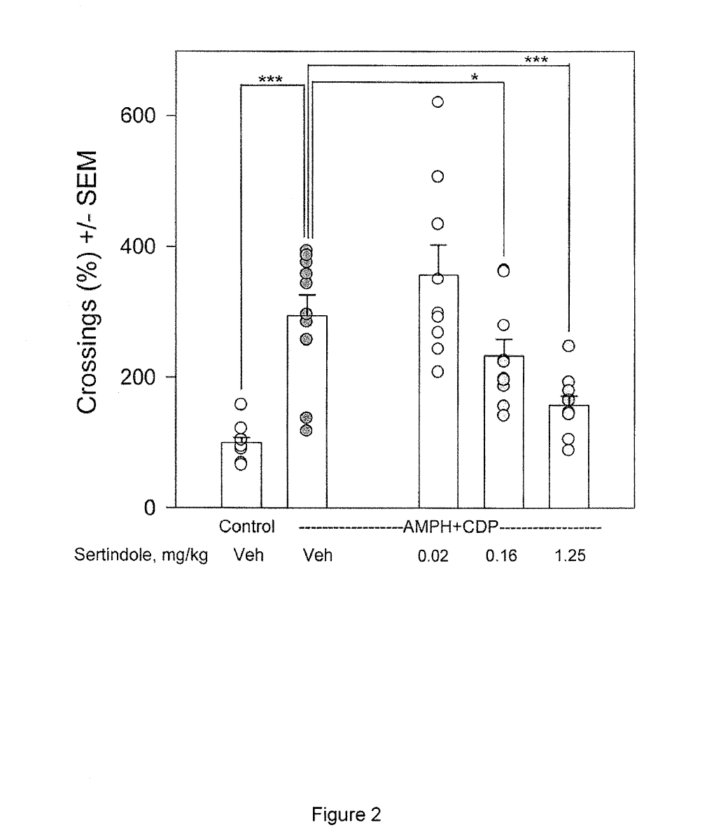

FIG. 2: Pre-treatment with sertindole (about 120 min, s.c.) reduced the induced locomotor activity. The effect was significant at about 0.16 mg/kg (*p<0.05 vs. AMPH−CDP group) and about 1.25 mg/kg (***p<0.001 vs. AMPH−CDP group).

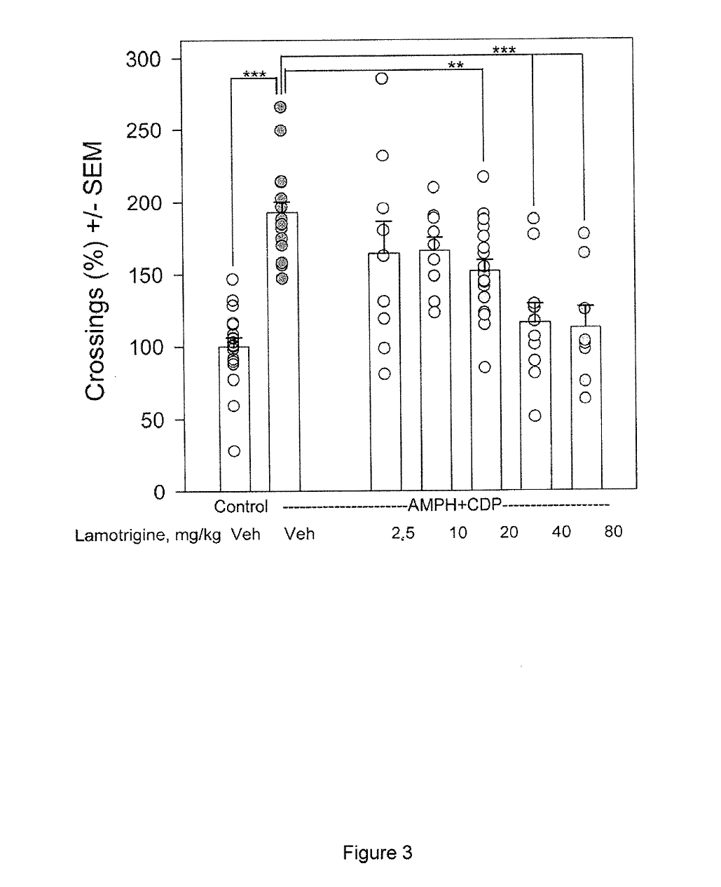

FIG. 3: Pre-treatment with lamotrigine (about 30 min, s.c.) reduced the induced locomotor activity. The effect was significant at about 20 mg/kg (**p<0.01), about 40 mg/kg and about 80 mg/kg (***p<0.001).

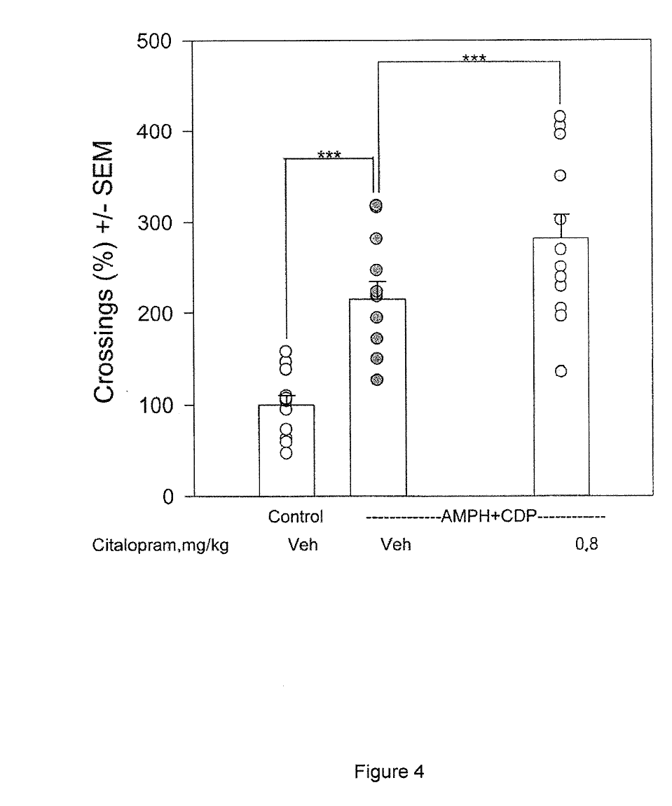

FIG. 4: Pre-treatment with citalopram (about 30 min, s.c.) produced an increase in locomotor activity (***p<0.001 vs. AMPH−CDP group)

Results: In all experiments, AMPH+CDP co-administration induced a significant increase (about 191-295%) in locomotor activity (P's<0.001). Lithium chloride significantly decreased AMPH+CDP induced hyperactivity (F_{2, 61}=4.69, P=0.0 1). Post hoc Student-Newman-Keuls test showed that only the higher lithium dose (about 0.9 mEq/kg) significantly inhibited the AMPH+CDP induced locomotor activity (P<0.003). Lamotrigine also significantly and dose-dependently decreased the AMPH+CDP induced locomotor activity (F_{5,137}=2.92, P<0.05). Post hoc analysis showed that the doses of about 20 mg/kg, about 40 mg/kg, and about 80 mg/kg significantly counteracted the AMPH+CDP induced hyperactivity (P<0.04, P<0.001, and P<0.001, respectively). Interestingly, sertindole significantly decreased the AMPH+CDP induced locomotor activity. Accordingly, the present data supports the anti-mania potential of sertindole.

As previously described, while clinical studies demonstrate the effectiveness of sertindole to treat schizophrenic patients, the instant experiments indicate that a lower effective dose of sertindole to treat mania and/or bipolar disorders may be needed as compared to the effective dose needed to elicit an anti-psychotic effect. Accordingly, the maintenance dose needed to treat mania appears be lower than the dose needed to elicit an anti-psychotic response.

Table 1: The administered doses of lithium, sertindole, lamotrigine and citalopram had no significant effect on baseline locomotor activity of control rats subjected to locomotor box for about 2 hours.

TABLE 1

| | Average Crossings | | Average Crossings |
|---|---|---|---|
| Drug | in % (SEM) | | (SEM) |
| Control | 100.0 | (10.7) | 516.5 (55.4) |
| Lithium 0.2 mEq/kg | 137.1 | (15.0) | |
| Lithium 0.9 mEq/kg | 121.1 | (10.3) | |
| Control | 100.0 | (7.5) | 322.3 (24.1) |
| Sertindole 0.02 mg/kg | 116.7 | (9.3) | |
| Sertindole 0.16 mg/kg | 119.9 | (9.8) | |
| Sertindole 1.25 mg/kg | 132.4 | (11.0) | |
| Control | 100.0 | (10.2) | 435.0 (44.1) |
| Citalopram 0.8 mg/kg | 71.0 | (3.5) | |
| Control | 100.0 | (6.1) | 459.5 (50.0) and |
| | | | 458.6 (23.4)^{#} |
| Lamotrigine 2.5 mg/kg | 114.5 | (13.8) | |
| Lamotrigine 10 mg/kg | 107.9 | (8.6) | |
| Lamotrigine 20 mg/kg | 97.2 | (6.6) | |
| Lamotrigine 40 mg/kg | 82.0 | (9.9) | |
| Lamotrigine 80 mg/kg | 83.0 | (6.3) | |

### Example 2

### Sensitized AMPH Response Model

Subjects: Male Crl:NMRI mice (about 20 g) were at the animal facility about 5 days prior to the start of the experiment and were housed in climate-controlled animal facilities under normal light-dark cycle (lights on about 06:00 to about 18:00). The mice were kept 6 per cage and allowed enrichment (two plastic houses+nesting material).

Drugs: All compounds were administered subcutaneously and are listed as free base doses. The mice were pre-treated with either d-amphetamine (about 1.8 mg/kg) or vehicle (about 0.9% NaCl, about 10 ml/kg) once daily for five consecutive days.

Test procedure: About 17-19 days after the pre-treatment, the animals were treated with test substance or vehicle and individually placed to habituate for about 30 min in motility boxes (about 20×about 32 cm) equipped with about 5×about 8 light sources and infrared cells spaced by about 4 cm. The light beams crossed the cage 1.8 cm above the bottom of the cage. Recording of a motility count requires interruption of adjacent light beams, thus avoiding counts induced by stationary movements of the mice. After habituation, an acute dose of d-amphetamine (about 0.95 mg/kg) or vehicle was administered and data acquisition was begun and lasted for about 30 min.

Data Analysis: Data were averaged within the groups (n=12) and are presented as mean±SD. The acute effect of test substance against the sensitized response to amphetamine was statistically evaluated by one-way ANOVA of variance: Dunn's method was applied for post hoc testing against the sensitized group receiving (NaCl, about 1 ml/kg) followed by CDP (about 8.9 mg/kg in 10% hydroxypropyl-beta-cyclodextrin) was administrated about 35 minutes before the test. A parallel set of control animal received two vehicle injections.

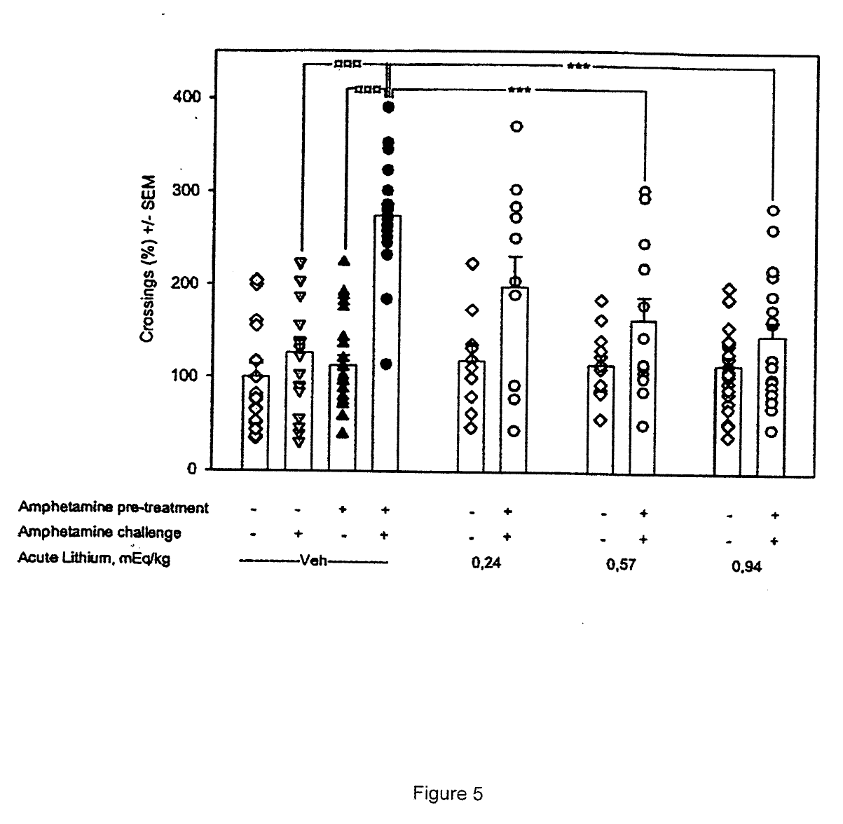

FIG. 5: Pre-treatment with lithium (about 60 min, s.c.) attenuated the induced hyperactivity. The effect of lithium was significant at about 0.57 and about 0.94 mEq/kg (p<0.001). The tested lithium doses had no significant effect on activity.

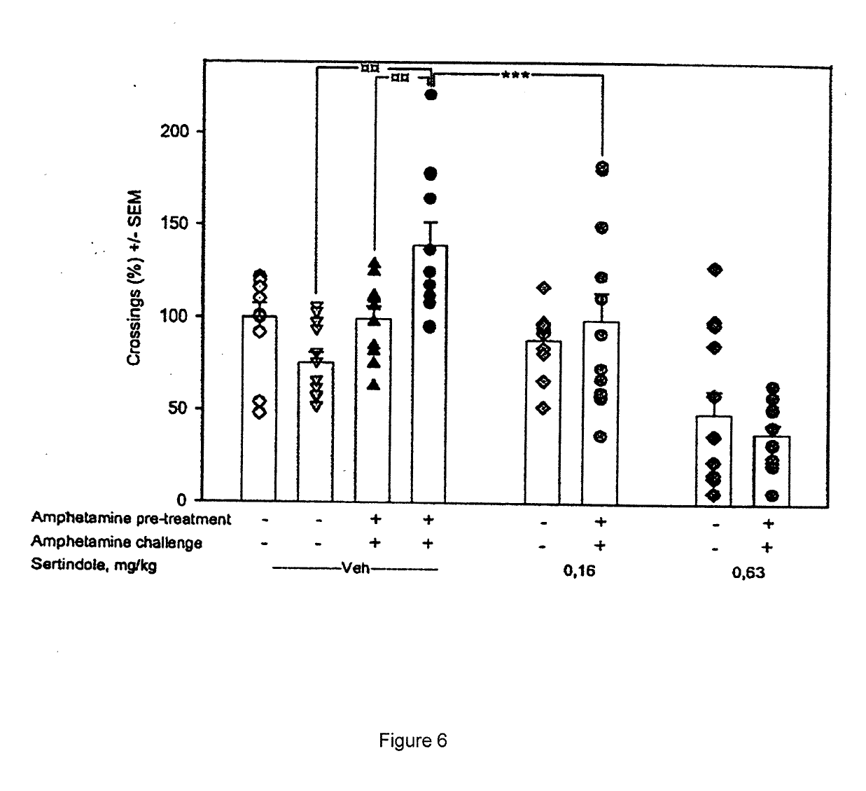

FIG. 6: Pre-treatment with sertindole reversed the induced hyperactivity in both tested doses, however, sertindole (about 0.63 mg/kg) significantly decreased the saline activity. Thus, sertindole at 0.16 mg/kg significantly reversed the induced hyperactivity.

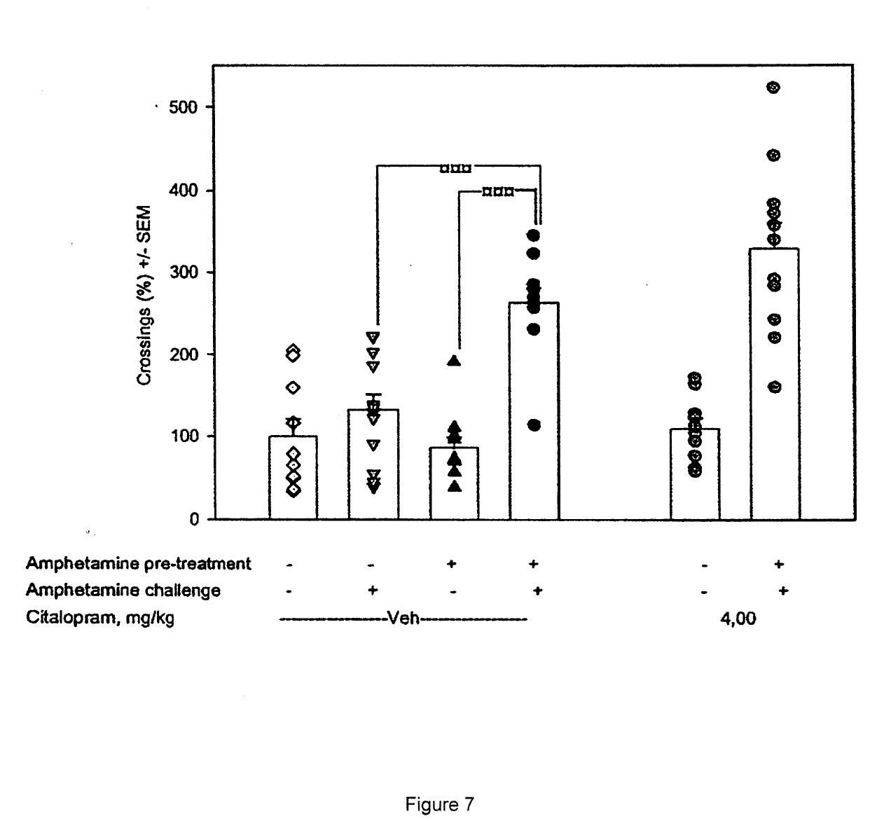

FIG. 7: Pre-treatment with citalopram (about 30 min, s.c.) had no effect on the induced hyperactivity.

Results: In all experiments, AMPH co-administration induced a significant increase in locomotor activity. Lithium chloride and sertindole significantly decreased AMPH+CDP induced hyperactivity. Citalopram was inactive. Additionally, pre-treatment with lamotrigine, carbamazepine, valproate and haloperidol reversed the effect of acute AMPH administration (Husum, H. 8^{th} World Congress of Biological Psychiatry 2005, poster no. 32.06). Accordingly, the present data supports the anti-mania potential of sertindole.

While clinical studies demonstrate the effectiveness of sertindole to treat schizophrenic patients, the instant experiments indicate that a lower effective dose of sertindole to treat mania and/or bipolar disorders may be needed as compared to the effective dose needed to elicit an anti-psychotic effect. Accordingly, the maintenance dose needed to treat mania may be lower than that needed to elicit an anti-psychotic response.

1. A method of treating mania comprising administering to a subject in need thereof an anti-manic effective dose of sertindole.

2. A method of treating bipolar disorders comprising administering to a subject in need thereof an anti-manic effective dose of sertindole.

3. A method of treating mania in a subject suffering from a bipolar disorder, Alzheimer's disease, neurosyphilis, stroke, midbrain infarctions, thalamic infarctions, bilateral thalamic infarction, Huntington's disease, Sydenham's chorea, Chorea gravidarum, Cushing's disease, thyrotoxicosis, cerebral tumors, multiple sclerosis, systemic lupus erythematosus, vitamin B12 deficiency, hepatic encephalopathy, uremia, Creutzfeldt-Jakob disease, ictal mania and post-ictal psychoses comprising administering to said subject in need thereof an anti-manic effective dose of sertindole.

4. The method of claim 2 or 3, wherein the bipolar disorder is bipolar I disorder, bipolar II disorder, cyclothymia or bipolar disorder NOS.

5. The method of anyone of claims 1, 2 or 3, wherein the subject is a human patient diagnosed with mania or a bipolar disorder.

6. The method of anyone of claims 1, 2 or 3, wherein the anti-manic effective dose of sertindole is a maintenance dose.

7. The method of claim 6, wherein the maintenance dose is from 1-12 mg/day.

8. The method of claim 7, wherein the maintenance dose is from 2-11 mg/day.

9. The method of claim 8, wherein the maintenance dose is from 3-10 mg/day

10. The method of claim 9, wherein the maintenance dose is from 5-8 mg/day.

11. The method of claim 10 wherein the maintenance dose if from 6-7 mg/day.

12. The method of anyone of claims 1, 2 or 3, wherein the subject to be treated does not respond sufficiently to current therapies with an anti-psychotic.

13. The method of claim 12, wherein the anti-psychotic is selected from the group consisting of olanzapine, risperidone, clozapine and haloperidol.
